(19) United States
(12) Patent Application Publication
(10) Pub. No.: US 2011/0144565 A1
(43) Pub. Date: Jun. 16, 2011

(54) COMBINATION OF A TILIROSIDE AND A PEPTIDE

(75) Inventor: Olivier Courtin, Neuilly Sur Seine (FR)

(73) Assignee: LABORATOIRES CLARINS, Cergy Pontoise Cedex (FR)

(21) Appl. No.: 12/999,971

(22) PCT Filed: Jun. 18, 2009
(86) PCT No.: PCT/FR2009/000732

§ 371 Date: Feb. 24, 2011

(30) Foreign Application Priority Data
Jun. 19, 2008 (FR) 0803425

(51) Int. Cl. A61M 37/00; A61K 38/05; A61P 17/02; A61Q 19/08

(52) U.S. Cl. 604/20; 514/21.91; 514/18.8; 604/22

(57) ABSTRACT

The invention relates to a combination including at least one bioflavonoid, in particular tiliroside, and at least one peptide, in particular palmitoyl Lysine-Valine-5 diaminohydroxybutyrate. The invention also relates to a cosmetic or dermatological composition containing said combination.

### CROSS REFERENCE TO RELATED APPLICATIONS

This application is a 35 U.S.C. §371 National Phase Entry Application from PCT/FR2009/000732, filed Jun. 18, 2009, and designating the United States, which claims priority under 35 U.S.C. §119 to French Patent Application No. 08 03425 filed Jun. 19, 2008, which is incorporated herein in its entirety.

### FIELD OF THE INVENTION

The present invention relates to a combination comprising at least one bioflavonoid, in particular tiliroside, and at least one peptide, in particular palmitoyl lysine-valine-5 diaminohydroxybutyrate. The present invention also relates to a cosmetic or dermatological composition containing said combination, and also to the use thereof for preventing, delaying or combating skin aging and/or the appearance of the signs of skin aging. In particular, this composition accelerates and/or stimulates the synthesis of collagen type I and also promotes skin cicatrization.

The subject of the present invention is also a method of cosmetically treating the skin.

### BACKGROUND OF THE INVENTION

In humans, from an anatomical point of view, the skin comprises two main parts. The thin superficial part, which is called the epidermis, is attached to a thinner internal part, the dermis.

The epidermis (etymologically formed in Greek from the words epi, on, and derma, skin) denotes the tissue of epithelial nature which covers the dermis. The epidermis is composed mainly of three types of cells: keratinocytes, melanocytes and Langerhans cells. The epidermis is not irrigated by any blood vessel. The cells which make up the epidermis are fed by diffusion from the dermis. On the other hand, the epidermis contains many nerve endings.

The dermis is clearly separated from the epidermis by the “dermal-epidermal junction” and beneath said dermis lies, without any clear-cut limit, the hypodermis.

The dermis is made up of various cell types, and in particular fibroblasts, responsible for synthesizing and maintaining the extracellular material. They are cells of mesenchymal origin which synthesize collagen, elastin, ground substance and structural glycoproteins. Their activity is intense during cicatrization phenomena.

The dermis is made up of a connective tissue which combines collagen fibers, elastic fibers and various cells bathing in an amorphous ground substance. It also contains the cutaneous appendages, which are the pilosebaceous follicles and the sweat glands, and also vessels and nerves. Through its fibers and its ground substance, the dermis contributes first of all to giving the skin its mechanical properties: elasticity, impact resistance, etc.

The fibers of the dermis comprise collagen fibers and elastic fibers. They represent quantitatively the most predominant structural proteins of the dermis, i.e., respectively, 75% and 5% of their dry weight. Their relative proportion and their arrangement are different according to the superficial or deep regions of the dermis.

The collagens form a very large family. They are extracellular matrix molecules composed of three polypeptide chains bearing the following 3 amino acid repeat:
Gly-X-Y, where X and Y are often prolines and hydroxyprolines.

The “collagen fibers” of the dermis are respectively made up of collagen I and collagen III, around an axis composed of collagen V. These collagens belong to the fibrillar collagen group. In adults, collagen I is, on average, six times more abundant than collagen III. The proportion of collagen I increases toward the hypodermis.

The collagen I/collagen III ratio decreases during aging. An exponential increase in chemical bridges between the collagen fibers, due to the nonenzymatic Maillard glycation reaction, is observed. This chemical bridging of the aged collagen results in increasing rigidification of the fibers, which makes them more resistant to attack by collagenases and by free radicals. Its degradation and its renewal are thus slowed down.

The fibroblast is a key cell of connective tissue which is involved in the formation and stabilization of the elastic fibers, but also in the dystrophy and lysis of said fibers. During aging, the fibroblast decreases its activity and this resting cell is often called a fibrocyte. It becomes globular, with a decrease in its cytoplasm and increased scarcity of its endoplasmic reticulum, the vesicles of which are very disperse. The cytoskeleton takes on a fascicled appearance. This cell is no longer in contact with the collagen.

### SUMMARY OF THE INVENTION

On reading the above, the importance of collagen in the structure of the dermis and, consequently, the need to stimulate and accelerate its synthesis in order to prevent, delay or combat skin aging, and in order to promote cicatrization processes, is understood.

The objective of the present invention is to provide a product which makes it possible to stimulate and/or accelerate the synthesis of collagen type I in a mammal, and in particular in humans.

Another objective of the present invention is to provide a product which makes it possible to preventively or curatively treat, in a mammal in general, and in particular in humans, skin aging and/or the appearance of the signs of skin aging.

Recently, dermatological techniques for esthetic purposes, such as peels, lasers, pulsed flash lamps, radiofrequency treatments, LED treatments, etc., have been developed for the prevention or treatment of skin aging.

These cosmetic or dermatological treatment techniques can generate a skin abrasion which then requires cicatrization and/or neosynthesis of fibers for dermal restructuring effects for anti-aging purposes.

An objective of the present invention is the provision of a product which promotes cicatrization of the skin of a mammal, and in particular of a human being.

An objective of the present invention is also the provision of a product which makes it possible to promote collagen neosynthesis and to optimize skin remodeling in a mammal, and in particular in a human being, during a cosmetic and/or dermatological treatment selected from the treatments mentioned above.

Yet another objective of the present invention is the provision of a method of cosmetically treating the skin for preventing, delaying or combating the appearance of the signs of aging and/or age-related skin damage and/or for promoting collagen neosynthesis and/or for optimizing skin remodeling during a dermatological treatment selected from a laser treatment, a pulsed flash lamp treatment, a radiofrequency treatment, an LED treatment, a peel and a microdermabrasion.

### BRIEF DESCRIPTION OF THE DRAWINGS

FIG. 1 graphically depicts results obtained with the invention.

### DETAILED DESCRIPTION OF THE INVENTION

In order to achieve these objectives, the applicant has discovered a combination comprising at least one bioflavonoid, more particularly tiliroside, and at least one peptide, palmitoyl lysine-valine-5 diamino-hydroxybutyrate or Palm-Lys-Val-Dab-OH, which has advantageous properties.

The Palm-Lys-Val-Dab-OH peptide is known in the prior art for its biological actions such as the stimulation of laminin V synthesis and the stimulation of collagen I synthesis. It is described for its uses as an anti-aging, anti-wrinkle and restoring active agent.

Tiliroside has already been described in the prior art as an anti-aging active agent for sensitive skin and as an agent which protects against oxidative stress and microinflammation processes. It is also known for its anti-allergic, anti-elastase and anti-collagenase properties.

Thus, in document US 2004/0081675, tiliroside is described and claimed as a UV-screening agent, an antioxidant, a free-radical scavenger, an active agent against oxidative stress, an anti-aging active agent, an anti-allergic active agent, an anti-inflammatory active agent and an active agent for the stabilization of UV-screening agents.

Document EP 1 393 733 describes tiliroside for the treatment of atopic eczema. Furthermore, the use of a composition containing tiliroside for treating pathological conditions affecting collagen is mentioned.

However, none of these documents mentions or suggests that tiliroside would have a collagen I synthesis-stimulating activity.

The combination comprising tiliroside and palmitoyl lysine-valine-5 diaminohydroxybutyrate, and also its collagen synthesis-stimulating activity, have never been described.

Specifically, surprisingly, it has been discovered by the applicant that the combination of tiliroside and palmitoyl lysine-valine-5 diaminohydroxybutyrate has a collagen I synthesis-stimulating activity, and that this activity is much greater than the sum of the effects of each of these active agents taken separately.

A noteworthy property of the combination of the present invention is that it exhibits effects in greater proportions than those reasonably expected from the simple addition of the effects of each of these compounds taken separately.

An advantage of this property is that it allows the use, in a cosmetic or dermatological composition, of an amount of each of these products which is less than what is generally accepted to be used.

Thus, a subject of the present invention is a combination comprising at least one bioflavonoid, tiliroside, and at least one peptide, palmitoyl lysine-valine-5 diaminohydroxybutyrate.

Tiliroside (C₃₀H₂₆O₇) is a known compound which is commercially available. For the implementation of the present invention, it can be used pure or in the form of a plant extract. It is sold by Merck KgaA in a more than 95% purified form. Document WO 2006/099930 describes the preparation of tiliroside from plants.

Palmitoyl lysine-valine-5 diaminohydroxybutyrate is sold by the company Pentapharm in solution at 0.2% in a water/glycerol solvent.

Preferably, the percentage by weight of tiliroside (T) relative to the palmitoyl lysine-valine-5 diamino-hydroxybutyrate (P), T/P, is between 0.1 and 1000, advantageously between 0.25 and 200.

A subject of the present invention is also a cosmetic and/or dermatological composition containing a combination as described above in a cosmetically or dermatologically acceptable carrier.

The cosmetically acceptable carriers, i.e. carriers compatible with the skin, are in all the forms known to those skilled in the art and normally used for topical application, in particular in the form of an oil-in-water or water-in-oil emulsion, an aqueous, oily or aqueous-alcoholic solution, an aqueous or oily gel, a liquid, pasty or solid anhydride product, or a dispersion of oil in an aqueous phase.

The composition of the invention may also contain adjuvants normally used in the cosmetics and dermatological fields, such as hydrophilic or lipophilic gelling agents, hydrophilic or lipophilic active agents, preservatives, antioxidants, solvents, fragrances, screening agents, fillers, pigments, chelating agents and dyes.

The composition of the invention may be in the form of creams, gels, sera, lotions, milks or oils. It may, where appropriate, be applied to the skin the form of an aerosol. It may also be in solid form, for example in the form of a stick.

Preferably, this composition contains 0.01% to 10% by weight of tiliroside and 0.0001% to 1% by weight of palmitoyl lysine-valine-5 diaminohydroxybutyrate. Advantageously, the composition contains 0.01% to 1% by weight of tiliroside and 0.005% to 0.5% by weight of palmitoyl lysine-valine-5 diaminohydroxybutyrate. Even more advantageously, the composition contains 0.05% to 0.5% by weight of tiliroside and 0.001% to 0.1% by weight of palmitoyl lysine-valine-5 diaminohydroxybutyrate.

The composition according to the present invention is used for stimulating and/or accelerating the synthesis of collagen type I. This stimulation and/or acceleration of the synthesis of collagen I is due to the synergistic effect of the combination of tiliroside and palmitoyl lysine-valine-5 diaminohydroxybutyrate.

This property of the composition of the present invention can be used in all circumstances where collagen neosynthesis is desired.

Consequently, the composition according to the present invention can be used for preventing, delaying or combating skin aging and/or the appearance of the signs of skin aging.

In the context of the invention, the expression “signs of skin aging” is intended to mean all the modifications of the external appearance of the skin due to chronobiological or photoinduced aging. These signs of skin aging are, for example, the appearance of wrinkles and fine lines, of slackening of the skin, of weathered skin, of thinned skin and of dull and/or lifeless skin, and a lack of skin elasticity and/or tonicity. Among the signs of skin aging, mention may also be made of any inner modifications of the skin which do not systematically result in a modified external appearance, for example any inner degradations of the skin, and in particular collagen degradation.

In addition, the appearance of stretch marks due to the rupturing of collagen fibers and elastic fibers following, in particular, too rapid and too abrupt a stretching of the skin, can also be prevented and/or treated with the composition of the invention.

Stretch marks frequently appear during pregnancy or following a substantial change in weight or even rapid growth.

Some cosmetic and/or dermatological treatments for esthetic purposes can induce a skin abrasion requiring cicatrization and/or collagen neosynthesis in order to optimize skin remodeling. Among these treatments, mention may be made of a laser treatment, a pulsed flash lamp treatment, a radiofrequency treatment, an LED (light-emitting diode) treatment, a peel and a microdermabrasion.

The objective of dermatological or cosmetic laser treatments is the prevention and treatment of skin aging.

Generally, various types of lasers are used according to the objective to be achieved. Irrespective of the type of laser used, a beneficial effect on skin remodeling is observed.

The principle of ablative lasers is to destroy very thin layers of skin. During the treatment, the laser light volatilizes the epidermis and the most superficial part of the dermis. These two thicknesses are thus eliminated. In the dermis, it is mainly the altered elastin fibers which are eliminated, but there is also a thermal effect which results in the formation of the new collagen fibers.

The lasers used here are CO₂ and/or erbium lasers.

The CO₂ laser (10 600 nm) used in continuous mode induces a skin abrasion with coagulation and is preferentially aimed at marked photoaging. The cicatrization requires approximately 7 days.

The pulsed Er:Yag laser (2950 nm), which is purely ablative (without coagulation, hence induction of bleeding), induces a more slight resurfacing and is aimed at less marked photoaging. The cicatrization is more rapid (eviction of approximately 7 days).

Remodeling lasers and rejuvenation lamps give more modest results, with virtually no side effects.

Remodeling lasers induce collagen neosynthesis without causing epidermal damage. They improve skin tonicity and texture and smooth out the relief (fine lines).

Use is made of devices of which the wavelength is preferably absorbed either by the water of the dermis, or by the superficial vessels of the dermis. Use may be made of lasers which emit in the infrared range (1064, 1320, 1450, 1540 nm) or in the visible range (pulsed dye laser, KTP laser) and pulsed lamps.

A new technique, fractional smoothing (fractional photothermolysis), can combine the advantages of the two methods above and of new techniques which act more specifically on sagging of the skin. This technique is based on a laser (1500 nm) which does not scan the entire surface, but makes impacts of 100 microns in diameter. At each session, 20% of the dermis is treated. The cicatrization is rapid (moderate erythema associated with a pseudo-tanned appearance).

Radiofrequency RF treatments (painful) and an infrared light instrument in the range of from 1100 to 1800 nm are used for preventing and/or treating sagging of the skin.

The peel is another nonsurgical technique which makes it possible to eliminate a precise and controlled skin thickness, and to induce a healthy regeneration of the destroyed layer and an anabolic stimulation of the underlying layers. Peels are classified as light, medium and deep according to the skin layer destroyed. The deeper the peel, the more serious the complications due to the peel.

Microdermabrasion comprises the projection of alumina hydroxide crystals and suction. The power of the suction, the projection speed and the number of passes over the same area can be adjusted so as to modulate the abrasion, which may be epidermal (exfoliation of the stratum corneum), superficial dermal (with or without bleeding) or dermal (with bleeding).

It is obvious from what is described above that all these techniques for cosmetic and/or dermatological purposes can cause varying degrees of skin abrasion which, subsequently, requires cicatrization.

The composition according to the present invention can be used for stimulating and/or promoting cicatrization.

In particular, it can be used for promoting or stimulating cicatrization after a cosmetic or dermatological treatment such as those described above. However, it can also be used for promoting or stimulating the cicatrization of a lesion due to an injury or to a surgical procedure.

The composition which is the subject of the invention can also be used for promoting collagen neosynthesis and for optimizing skin remodeling during a dermatological or cosmetic treatment selected from a laser treatment, a pulsed flash lamp treatment, a radiofrequency treatment, an LED treatment, a peel and a microdermabrasion.

In particular, this composition can be used as a cicatrizing product for accompanying treatments such as laser treatments, peels, microdermabrasion, etc. In this case, it is applied to the skin after the cosmetic or dermatological treatment and the application is renewed repeatedly with a frequency which can range from 1 to 5 times a day and until a satisfactory result is obtained.

The composition according to the present invention can also be used during one of the treatments mentioned above on the treated areas, for optimizing the performance levels of the treatments (for example: rejuvenation). The composition of the invention has a potentiating effect which reinforces the action of the dermatological or cosmetic treatment. In this case, the composition is applied to the area of skin to be treated just before (a few minutes or a few hours) the start of each cosmetic or dermatological treatment action.

The composition of the present invention can also be used alternately with each action and/or after the treatment in order to optimize the effectiveness of the esthetic treatments. The frequency and the duration of the treatment with this composition are adjusted according to the individual and to the cosmetic and/or dermatological treatment applied.

In addition, the composition of the invention can be used daily as an anti-aging or cicatrization-promoting product.

A subject of the present invention is also a method of cosmetically treating the skin for preventing, delaying or combating age-related skin damage and/or the appearance of the signs of skin aging, for promoting cicatrization and/or collagen neosynthesis and for optimizing skin remodeling during a dermatological treatment selected from a laser treatment, a pulsed flash lamp treatment, a radiofrequency treatment, an LED treatment, a peel and a microdermabrasion, said method comprising the application, to the skin, of the composition which is the subject of the present invention. This application can be carried out before the dermatological or cosmetic treatment, it can take place between two actions of this treatment or it can be subsequent to this treatment. This application to the areas of skin concerned can be repeated with a frequency and for a duration that those skilled in the art know how to adjust according to the individual and to the associated dermatological or cosmetic treatment.

Irrespective of the objective, a daily application, once or twice a day, for a period of a few days to several months, can be proposed.

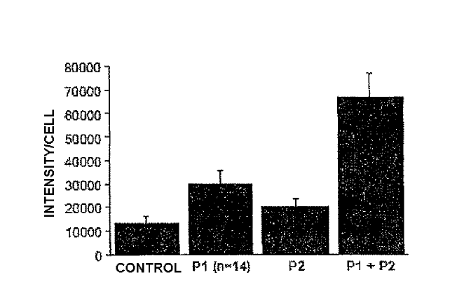

The invention will be understood more clearly and other characteristics thereof will emerge more clearly on reading the example which follows and which refers to FIG. 1 which represents the synergistic effect of the combination of tiliroside and palmitoyl lysine-valine-5 diaminohydroxybutyrate on the synthesis of collagen type I in normal adult human fibroblasts.

### Example 1

By visualizing the immunolocalization of collagen type I in normal adult human fibroblasts it was possible to demonstrate the synergistic effect of the combination of tiliroside and palmitoyl lysine-valine-5 diaminohydroxybutyrate on the synthesis of collagen type I, a major protein of the extracellular matrix of the dermis.

### Test Product:

We used 2×10⁻³% palmitoyl lysine-valine-5 diamino-hydroxybutyrate (P1), 5×10⁻⁴% tiliroside (P2), the combination of the two active agents (P1*P2) above and a control (C).

The fibroblasts were seeded into 6-well plates with a glass coverslip in a proportion of 70 000 cells per dish, in MEM (Minimum Essential Medium).

48 hours later, the cells were treated, or not treated (control), with the palmitoyl lysine-valine-5 diaminohydroxybutyrate or with the tiliroside or with the combination of the two active agents, and were incubated in an incubator for 72 hours at 37° C., 5% CO₂.

Each cell layer was then rinsed and fixed with methanol (−20° C.) before visualizing the collagen type I by immunofluorescence.

### Immunolocalization of Collagen Type I

The cells are incubated with the anti-collagen I primary antibody (Sigma, dilution 1:100) (source: mouse) and then with the anti-mouse secondary antibody coupled to TRITC (Abcys, dilution 1:100). The incubation time between each antibody is 1 hour. The cell nuclei are visualized by means of Dapi labeling.

The coverslips are mounted on glass slides and then observed under a fluorescence microscope (Nikon Eclipse 50i). Photographs are taken of several fields of the cell population. The TRITC fluorescence images indicate the regions of localization of collagen type I. The DAPI fluorescence images indicate the cell nuclei and therefore the number of cells per field. The photographic images are analyzed using the Lucia image analysis software.

### Statistical Analysis and Results

The intensity of collagen labeling was observed on 14 and 15 photographic images according to the conditions.

This fluorescence intensity is related to the number of cells per image.

The differences in mean between the populations were studied by analysis of variance (Anova). This method uses measurements of variance in order to determine the significant or nonsignificant nature of the differences in mean measured on the populations.

The results obtained, presented in FIG. 1, show that palmitoyl lysine-valine-5 diaminohydroxybutyrate leads to a statistically significant (p<0.0001) increase in collagen produced by the human skin fibroblasts compared with the nontreated control.

Tiliroside stimulates, to a lesser extent but in a statistically significant manner (p=0.0015), the expression of collagen type I.

The simultaneous presence of palmitoyl lysine-valine-5 diaminohydroxybutyrate and of tiliroside leads to a greater stimulation of the synthesis of collagen type I than each of the constituents taken separately (significant interaction, p=0.0278).

This synergistic effect leads to an overexpression of collagen type I.

### Examples of Compositions

The peptide used in the examples of compositions which follow is palmitoyl lysine-valine-5 diaminohydroxybutyrate, in the commercial form supplied by the company Pentapharm in solution at 0.2% in a water/glycerol solvent.

The percentages are percentages by weight relative to the total weight of the composition.

### Cicatrizing W/O Emulsion

%

### GLYCEROL 5.00

### MAGNESIUM SULFATE 0.70

### ZINC SULFATE 0.35

### COPPER SULFATE 0.10

### GLYCOLS 5.00

### PEG 30 DIPOLYHYDROXYSTEARATE 4.00

### HYDROGENATED C16-C18 TRIGLYCERIDES 5.00

### PEG-45/DODECYL GLYCOL COPOLYMER 1.20

### C8 C10 TRIGLYCERIDES 19.00

### FLUID LIQUID PETROLEUM JELLY 8.00

### CERAMIDE 3 0.10

### ZINC OXIDE 4.00

### TILIROSIDE 0.10

### TOCOPHERYL ACETATE 0.20

### PEPTIDE 1.00

### DEMINERALIZED WATER q.s. 100

### Anti-Aging Gel

%

### POLYMERIC GELLING AGENT 1.00

### GLYCOLS 5.00

### TILIROSIDE 0.10

### PEPTIDE 1.00

### CHELATING AGENT 0.10

### PRESERVATIVE 0.10

### DEMINERALIZED WATER q.s. 100

### Anti-Aging O/W Emulsion

%

### CETEARYL GLUCOSIDE 3.50

### AE GLYCERYL MONOSTEARATE 2.50

### C8 C10 TRIGLYCERIDES 5.00

### SHEA BUTTER 3.00

### CETEARYL OCTANOATE 7.00

### CETYL ALCOHOL 0.30

### STEARYL ALCOHOL 0.30

### SILICONE 0.50

### PRESERVATIVE 0.10

### GLYCOLS 3.00

### VOLATILE SILICONE 5.00

### GLYCEROL 5.00

### TILIROSIDE 0.20

### PEPTIDE 1.00

### TOCOPHERYL ACETATE 0.20

### GELLING AGENT 0.30

### CHELATING AGENT 0.20

### DYE 0.06

### SODIUM HYDROXIDE q.s. pH

DEMINERALIZED WATER q.s. 100

1. A combination comprising tiliroside and palmitoyl lysine-valine-5 diaminohydroxybutyrate.

2. The combination as claimed in claim 1, in which the percentage by weight of tiliroside (T) relative to the palmitoyl lysine-valine-5 diamino-hydroxybutyrate (P), T/P, is between 0.1 and 1000, advantageously between 0.25 and 200.

3. A composition which is at least one of cosmetic or dermatological containing a combination as claimed in claim 1 in a cosmetically or dermatologically acceptable carrier.

4. A composition which is at least one of cosmetic or dermatological containing:
0.01% to 10% by weight of tiliroside;
0.0001% to 1% by weight of palmitoyl lysine-palm-lys-valine-5 diaminohydroxybutyrate.

5. The composition as claimed in claim 3, said composition being in a form selected from: a cream, a gel, a serum, a lotion, a milk and an oil.

6. The composition as claimed claim 3, for at least one of stimulating or accelerating synthesis of collagen type I.

7. The composition as claimed in claim 3, for preventing, delaying or combating at least one of skin aging or the appearance of the signs of skin aging.

8. The composition as claimed in claim 7, in which the signs of skin aging are selected from at least one of wrinkles, fine lines, sagging of the skin, weathered skin, thinned skin, dull skin or lifeless skin.

9. The composition as claimed in claim 3, for preventing or combating stretch marks.

10. The composition as claimed in claim 3, for promoting cicatrization.

11. The composition as claimed in claim 3, for promoting collagen neosynthesis and for optimizing skin remodeling during a dermatological or cosmetic treatment selected from: a laser treatment, a pulsed flash lamp treatment, a radiofrequency treatment, an LED treatment, a peel and a microdermabrasion.

12. A method of cosmetically treating the skin for preventing, delaying or combating at least one of age-related skin damage, appearance of the signs of aging, promoting collagen neosynthesis or for optimizing skin remodeling during a dermatological treatment selected from a laser treatment, a pulsed flash lamp treatment, a radio-frequency treatment, an LED treatment, a peel and a microdermabrasion, characterized in that the composition as defined in claim 3 is applied to the skin.

13. The composition of claim 4 containing 0.05-0.5% tiliroside.

14. The composition of claim 4 containing 0.001-0.1% palmitoyl lysine-palm-lys-valine-5 diaminohydroxybutyrate.
